(19) United States
(12) Patent Application Publication
(10) Pub. No.: US 2022/0217957 A1
(43) Pub. Date: Jul. 14, 2022

(54) UNIT, BUILDING AND METHOD FOR REARING INSECT LARVAE

(71) Applicant: Protifly, Mont-de-Marsan (FR)

(72) Inventors: Maxime Baptistan, Mont-de-Marsan (FR); Bastien Quinnez, Saint Cyr Au Mont D'Or (FR)

(21) Appl. No.: 17/609,111

(22) PCT Filed: May 7, 2020
(86) PCT No.: PCT/FR2020/050762

§ 371 Date: Nov. 5, 2021

(30) Foreign Application Priority Data
May 7, 2019 (FR) FR1904793

(51) Int. Cl. A01K 67/033

(52) U.S. Cl. CPC A01K 67/033

(57) ABSTRACT

The invention relates to a unit for farming insect larvae including at least one row (1001, 1002) of at least two stacked trays (1100, 1200, 1300), said unit being characterized in that said trays (1100, 1200, 1300) have farming surface areas of different dimensions and in that each tray comprises a means for thermoregulating its farming surface area, and adapted to the development stage of the larvae they contain.

### FIELD OF THE INVENTION

The invention relates to the field of insect larvae farming.

More particularly, the invention relates to an insect larvae farming unit, an insect larvae farming building comprising one or more units, and a method of farming insect larvae.

### PRIOR ART

As a result of population growth, the demand for protein to feed livestock is increasing while feed resources are becoming scarce. Consequently, the animal feed industry is increasingly turning to the farming of insect larvae in order to process them to recover proteins and lipids for use in animal feed.

However, industrial insect farming requires favorable environmental constraints to be met in order to ensure, on the one hand, the survival of the colony, and, on the other hand, satisfactory production yields. Thus, the temperature and the degree of hygrometry are two particularly important parameters for the larvae to develop in optimal conditions.

Since insects are ectothermic organisms, they do not have the ability to regulate their temperature and therefore depend on the external environment to regulate themselves. When feeding, the larvae release a lot of heat, which can be a risk to the colony if the temperature rises above 50°. Thus, during their development, the larvae can directly or indirectly release energy in the form of heat and emit ammonia gas (NH₃). Heat releases are responsible for changes in environmental conditions, both in terms of temperature and hygrometry in the farming area. These temperature variations can take the form of a thermal runaway that could jeopardize the health and even the survival of the colony. As regards to ammonia, it is a dangerous gas because it is irritating, corrosive and deflagrating, and it can affect the health of operators on the farming site and the safety and maintenance of the farming facilities. It is therefore necessary to control both the heat production and the gaseous atmosphere (hygrometry and ammonia) in order to guarantee optimal safety and farming conditions.

Among the prior art documents, document EP2144859 describes a facility for processing organic waste using insect larvae. The facility comprises a plurality of flat reaction vessels stacked on top of each other, and separated from each other by an air gap of a few cm. One of the side walls of the facility, adjacent to a side edge of the reaction vessels, called the ventilation wall, is provided with openings to the air spaces between the vessels. An air circulation system allows purified and thermoregulated air to be circulated from the ventilation wall openings of the facility through the air spaces between the vessels. A turbine also allows ammonia-contaminated air to be extracted. Document US2015223496 proposes a system for producing organic fertilizer and food from the processing of animal waste by an insect belonging to the Diptera order such as Musca domestica (housefly), Boettcherisca peregrine, and Tabanus. In this system, after the excrement has been converted into organic fertilizer, a fan is operated to blow heated air so that the insect larvae are dislodged from a first farming processing storage unit to a second storage unit opposite the first farming processing storage unit. In addition, such a system proposes thermoregulation by control of the air temperature, which is energy-consuming and unsuitable for intensive farming of insects.

Document EP2986107 describes a method and a system for farming insects using a plurality of individual crates filled, at least in part, with a substrate containing nutrient material and immature insect phases. The crates are installed in a climate zone comprising an aeration system. A conveyor system is used to collect the crates from the climate zone and return them thereto. Along the conveyor system are placed an observation system to obtain observations on the substrate and the larvae, and downstream thereof, a station for dispensing nutritive material. The method comprises the steps of aerating the nutrient substrate and the immature insect phases with the aeration system, when the crates are arranged in the climate zone, and periodically retrieving, using the conveyor system, at least one crate from the climate zone to send it to the observation system in order to obtain an observation of the substrate and the insect immature phases contained in said at least one crate, and to determine a requirement for adding an amount of supplementary nutrient feedstock before returning it to the climate zone or a reason to harvest the insect larvae.

Existing industrial facilities for farming insect larvae essentially consist of lining up and stacking plastic containers, the standard dimensions of which are usually of 60×40 cm, in a building. Such a stacking of containers saves space and maximizes production yields. The containers are moved during the larva development cycle to a feeding source, a control means, and a harvesting device, which are centralized. Existing facilities seek to optimize production volumes per square meter by over-densifying the farms on multiple vertical levels and a reduced surface area. However, such over-densification requires a very high energy consumption to maintain optimal environmental conditions for the development of the larvae. To this end, deconcentration of the ammonia atmosphere and maintenance of optimum temperature and hygrometry degree, in the room where the plastic containers are stored, are done by processing the ambient air and supplying new and thermoregulated air. Thus, the ambient air in the farming areas is constantly renewed and thermoregulated to reach a predetermined temperature and a predetermined hygrometry level. It is necessary to maintain the entire atmosphere of these areas in optimal conditions and therefore to heat or cool down the entire volume of the room in which the containers are stored, while bringing in a significant amount of fresh air in order to deconcentrate the atmosphere contaminated with ammonia. Consequently, the air treated in temperature, by heating or cooling down, and in hygrometry, by humidification or dehumidification, is directly evacuated to deconcentrate it from the ambient ammonia. The energy used to process the incoming air is therefore directly lost when it is exhausted. These installations are therefore very energy consuming.

Existing industrial insect larva farming facilities have the disadvantage of being over-densified and very energy-intensive in order to maintain optimal environmental conditions for colony development and to obtain optimum production yield. Furthermore, the farming containers, which are designed to be stacked on top of each other, are sized to contain larvae at the end of their cycle, so that a large surface area of the containers is not used during the first stages of larval development. This unused surface area nevertheless occupies a volume that is also thermoregulated. In addition, the side walls of the containers make air circulation more difficult, so that heat and humidity tend to stagnate in certain areas, making thermoregulation more difficult to achieve.

### Technical Problem

The aim of the invention is thus to overcome at least one of the drawbacks of the prior art.

The invention aims in particular to propose a simple and effective alternative solution for optimizing the farming surface area, in order to avoid having to thermoregulate a volume of air corresponding to unused surface areas. The invention also aims to provide a thermoregulation solution that is simple to implement, efficient and much less energy consuming than existing solutions. Finally, the invention also aims to facilitate and accelerate the farming process.

### BRIEF DESCRIPTION OF THE INVENTION

To this end, one object of the invention is a unit for farming insect larvae including at least one row of at least two stacked trays, said unit being characterized in that said trays have farming surface areas of different dimensions.

Preferably, the object of the invention is a unit for farming insect larvae including at least one row of at least two stacked trays, said unit being characterized in that said trays have farming surface areas of different dimensions and in that each tray comprises a means for thermoregulating its farming surface area. Preferably, said means for thermoregulating the farming surface operates mainly by radiation and even more preferably each tray is a tray thermoregulated by a heat transfer fluid.

Thus, the dimensions of the farming surfaces are adapted to the development stage of the larvae, so that the use of the farming surfaces of the trays is optimized. In particular, the trays have farming surface areas adapted to the stage of development of the larvae they accommodate, that is to say they have farming surface dimensions which increase as a function of the stage of development of the larvae they are intended to accommodate. This is a simple and effective solution to optimize the farming surface and allows not to thermoregulate the ambient air. In addition, the combination of increasing tray farming surface areas with a thermoregulation device integrated with the tray allows energy requirement to be reduced during farming, compared, for example, with systems encumbered by homogeneous farming surface areas limiting the diffusion of thermal regulation by convection.

According to other optional features of the insect larva farming unit, the latter may optionally include one or more of the following features, alone or in combination:
the trays have increasing farming surface areas and are arranged in a stepped manner, the tray with the smallest farming surface area being arranged at the top of the stack. Preferably, the trays are aligned on one side. This allows the transfer of larvae by gravity to be facilitated and the farming process to be optimized.
the ratio between the farming surface area values of two consecutive trays of the stack is between 1.2 and 2.2. Such ratio values have been identified as suitable for farming insects by stage of development. In particular, the length of the farming trays is identical while the width Iₙ of each tray is expressed according to the following formula: Iₙ=[(n)/N]*I_{N}, where N is the total number of trays, I_{N} is the width of the tray of rank N, Iₙ is the width of a tray of rank n, with n being between 1 and N, the tray of rank 1 being located at the top of the stack while the tray of rank N is located at the bottom of the stack. Thus, the farming surface area of each tray is sized so that it corresponds to the surface area necessary for the good development of the larvae according to their stage of development, during their growth cycle;
each tray is divided by removable dividers into as many parts as there are development stages of the insect larvae to be farmed, and the removable dividers are retracted as the stages of development of the larvae progress so that the farming surface area of the tray increases with the development stage of the larvae. This makes it possible to use only one size of tray, the farming surface area of which will be modulated according to the stage of development of the larvae that the tray is intended to receive. Thus, the farming surface area is optimized while standardizing the trays. Optimally, the removable divider can be arranged on the opposite side of the tray alignment length so as to promote the gravity transfer to the tray surface below.
The stacked trays of a same row are spaced at a height of a minimum distance of 10 centimeters, preferably at a height of a minimum distance of 20 centimeters, so as not to obstruct the circulation of the air flow favoring a better ventilation in order to extract any potential excess of humidity or gas such as ammonia generated by the farming process.
the unit comprises two rows arranged longitudinally opposite each other, separated by a floor, each row comprising at least two stacked trays. In particular, the unit comprises a movably mounted floor. Thus, there can be a significant gain in the footprint of the farming units and easier access to the lower tray.
each row comprises a stack of at least two trays, the last tray with the largest surface area being arranged on the ground. The last tray may, for example, be positioned under the movable floor separating the two rows facing each other.
each tray, located above the last lower tray of the stack, comprises a transfer device arranged to allow the transfer of larvae, by gravity, into the tray immediately below. Such a transfer device may be individual for each tray of the stack. A transfer device can be global and can be arranged to allow the transfer of larvae, by gravity, into the tray immediately below of several farming units. This speeds up and automates the farming process.
each tray, located above the last lower tray of the stack, comprises a movable wall arranged to allow the transfer of larvae, by gravity, into the tray immediately below. Such a movable wall, preferably a movable side wall, allows the farming surface area to be optimized according to the stage of development of the insect, the energy consumption for thermoregulation to be limited to the used surfaces only, this thermoregulation to be adjusted independently for each tray, and the farming process to be sped up and automated.
the movable wall comprises closing means, for example magnetized closing means, ensuring the farming surface is sealed when said movable wall is in a closed position. This allows the growth phases to be well segmented for homogeneity of the larvae harvested at the end of the process.
each tray, located above the last lower tray of the stack, further comprises a translatably movable scraper, capable of transferring larvae to a tray located just below in the stack. In particular, the translatably movable scraper is adapted to transfer larvae to a tray located just below in the stack when the movable wall is in the open position. A movable scraper according to the invention speeds up and automates the farming process.
the last tray of the stack comprises a mechanized harvesting device, preferably translatably movable along the longitudinal axis of said last tray.
each tray comprises a plurality of farming modules connected to each other along the longitudinal axis of said tray. Thus, it is possible to optimize the farming surface area and to establish large-scale farming while controlling individually each part of the farming.
each tray may be comprised of a plurality of modules connected to each other along the longitudinal axis of said tray. Thus, it is possible to optimize the farming surface area, to establish large-scale farming adaptable to the targeted production volume while limiting the assembly time of a farming line.
each constituent module of each tray located above the last tray of the stack comprises an elementary scraper, said elementary scrapers of a tray being synchronized with each other to form said scraper of said tray. This speeds up and automates the farming process.
each constituent module of each tray located above the last tray of the stack comprises an elementary movable side wall, said elementary movable walls of a tray being synchronized with each other to form said movable side wall of said tray. This speeds up and automates the farming process.
the last tray of the stack comprises a scraper-suction device, preferably translatably movable along the longitudinal axis of said tray, connected to a suction system via a network of hoses. Such a movable scraper-suction device speeds up the harvesting process.
the unit comprises at least one temperature measuring device, preferably configured to measure the temperature of the farming surface of each tray. This allows the farming conditions to be improved by measuring/detecting more precisely the temperature variations and thus an optimal farming temperature to be maintained.
the temperature measuring device corresponds to a temperature probe, each tray comprises at least one temperature probe. This improves the farming conditions. In particular, the temperature measuring device corresponds to a temperature probe, each tray comprises at least one temperature probe, and a means for thermoregulating its farming surface area. These elements, as will be described, allow energy requirements to be reduced during farming compared to systems where the entire air volume is thermoregulated, each tray to be thermoregulated independently according to real needs, and farming conditions to be improved.
the thermoregulation means comprises at least one pipe arranged linearly and configured to allow the flow of a heat transfer fluid, said pipe being arranged under the farming surface, said heat transfer fluid allowing a transfer of energy with the farming surface. This makes the installation less energy consuming.
the thermoregulation means comprises pipes arranged linearly and configured to allow the flow of a heat transfer fluid, said pipes being arranged in a cavity, provided in the tray and filled with a heat-conducting liquid or material, said heat transfer fluid allowing a transfer of energy with the farming surface via said heat-conducting liquid or material. This makes the installation less energy consuming.
the thermoregulation means comprises linearly arranged pipes configured to allow the flow of a heat transfer fluid, said pipes being covered by a concrete topping forming the farming surface with which the heat transfer fluid performs an energy transfer. Thus, the process consumes a smaller amount of energy.
the thermoregulation means comprising pipes covered with a concrete topping is preferably installed on the lower farming tray placed on the ground;
the thermoregulation means comprising pipes arranged in a cavity filled with a heat-conducting liquid or material is preferably installed on the farming trays located above the lower farming tray of the stack.
the opening and closing of the movable wall of each tray are controlled by a larva transfer control device, the control of the opening of the movable wall and of the scraper being carried out as a function of temperature values measured on the farming surface of each tray, of an amount of feedstuff dispensed, and as a function of time.
the side walls of each tray are provided, on their upper end located opposite the farming surface of the tray, with an inverted “U”-shaped rim. This rim advantageously allows the crawling larvae to fall by gravity onto the farming surface, thus avoiding the escape thereof.

The invention also relates to a building for farming insect larvae, characterized in that it comprises at least one insect larva farming unit as described above.

According to other optional features of the building, the latter may optionally include one or more of the following features, alone or in combination:
it further comprises a tank for dispensing larva feedstuff and a feed control device, configured to control the conveyance of the contents of said tank to the trays of each unit as a function of time and/or the quantity of larvae and/or their nutritional requirements. Thus, this speeds up the farming process.
it comprises a thermoregulation control device configured to control a thermoregulation means of each tray of each unit, independently, as a function of temperature values measured on the farming surface of each tray and of flow rate values of the heat transfer fluid flowing in the pipes of said thermoregulation means. This makes the building's operation less energy consuming.
it further comprises a larva transfer control device, configured to control the opening and closing of a movable wall of one or more trays of each unit and to control, in a manner synchronized with the opening of the movable wall, a transfer device and the control of the opening of the movable wall and of the transfer device is carried out as a function of the development stage of the larvae, of temperature values measured on the farming surface of each tray, of the amount of feedstuff dispensed, and as a function of the time. The transfer of the larvae may also involve the transfer of the substrate.
it further comprises a larva transfer control device, configured to control the opening and closing of a movable wall of one or more trays of each unit and to control, in a manner synchronized with the opening of said movable wall, a scraper of the corresponding tray and the control of the opening of the movable wall and of the scraper is carried out as a function of temperature values measured on the farming surface of each tray, of the amount of feedstuff dispensed, and as a function of time. The transfer of the larvae may also involve the transfer of the substrate.
it further comprises a device for controlling the harvesting of mature larvae which is capable, on the one hand, of actuating a suction system coupled to a hose or to a network of hoses connected to a scraper-suction device of a lower tray and, on the other hand, of actuating said scraper-suction device in translation along the longitudinal axis of said associated lower tray. Thus, this speeds up the harvesting process.
it further comprises an aeration control device capable of activating the operation of extractors and the opening or closing of a reclosable air inlet, as a function of values recorded by at least one device for measuring temperature, the concentration of ammonia in the gas phase, and the hygrometry level. Thus, the farming conditions are improved and it speeds up the farming process.
the devices for controlling thermoregulation, for controlling the transfer of larvae, for controlling the harvesting of larvae, for controlling aeration and/or for controlling feeding are combined in a single automatic supervision device.

Finally, the invention relates to a method of farming insect larvae implemented in at least one previously described farming unit, characterized in that it comprises the following steps:
placing insect larvae on a first farming tray, located at the top of the stack of trays and having a first farming surface, and dispensing larva feedstuff in said first farming tray in at least one dose corresponding to the development stage of said larvae, preferably by means of a feeder tank and at regular intervals by means of a feeding control device,
after a predetermined period of time corresponding to a first farming stage, transferring the larvae (that is to say with the substrate) from the first tray to a second tray located immediately below in the stack and having a second farming surface area greater than the first farming surface area,
dispensing the feedstuff for said larvae in at least one dose corresponding to their development stage, preferably by means of said feeder tank and at regular intervals, and waiting for the end of a second predetermined period of time corresponding to a second development stage, and repeating the steps of transferring and feeding the larvae until the larvae have been transferred and fed in the last tray located at the bottom of the stack,
when the larvae have reached their last development stage, harvesting at least said larvae (that is to say with their substrate).

Such a process is quicker to implement, less energy consuming than prior art processes, and allows for optimization of the farming surface area.

Other features and advantages of the invention will become apparent upon reading the description given by way of illustrative and non-limiting example, with reference to the appended figures, which represent:

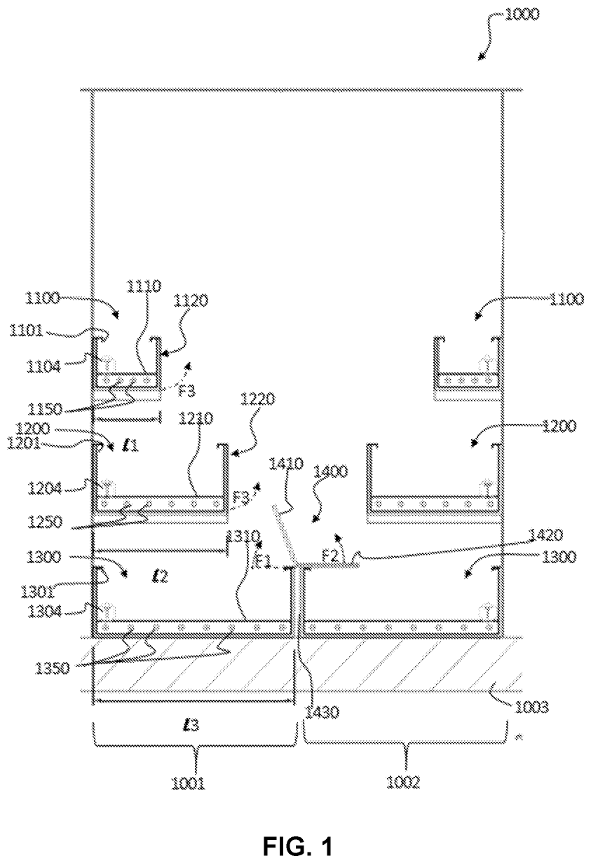

FIG. 1, a cross-sectional diagram of an insect larva farming unit according to a preferred embodiment of the invention,

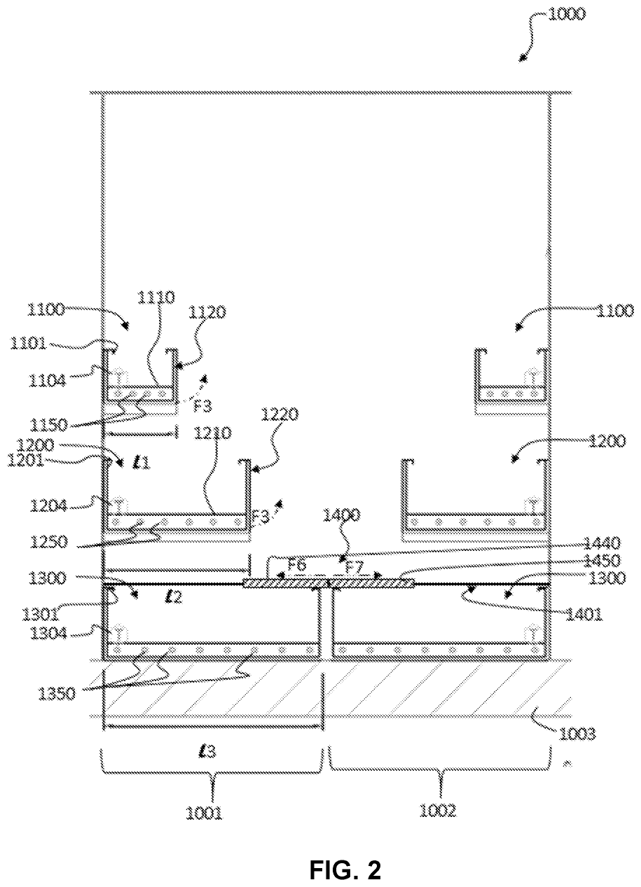

FIG. 2, a cross-sectional diagram of an insect larva farming unit where a movable floor is made in an embodiment different from that shown in FIG. 1,

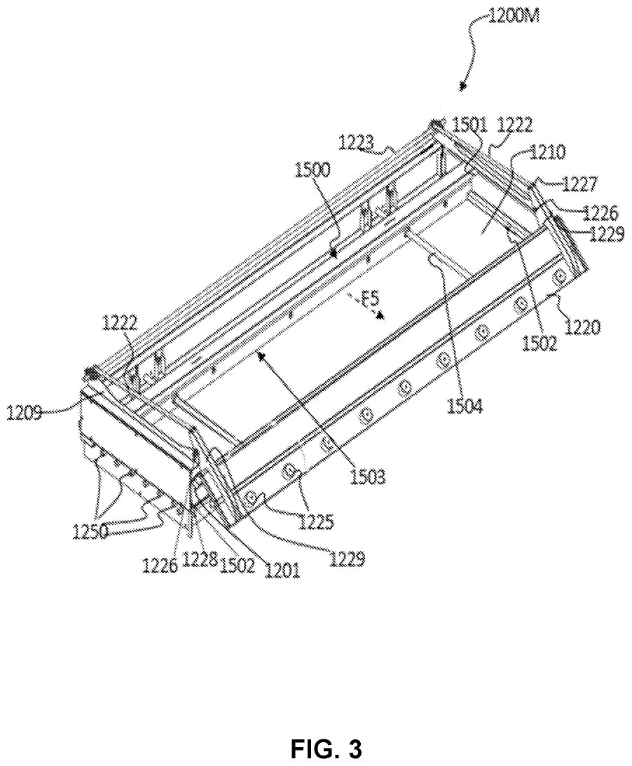

FIG. 3, a perspective diagram of a farming module for making a farming tray according to one embodiment, said module being equipped with an elementary scraper and an elementary movable wall,

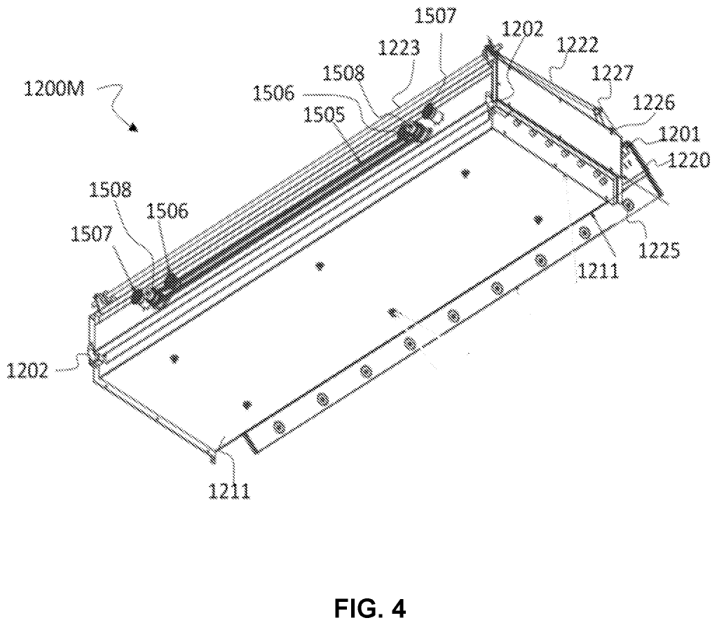

FIG. 4, a perspective and bottom view diagram of the module in FIG. 3,

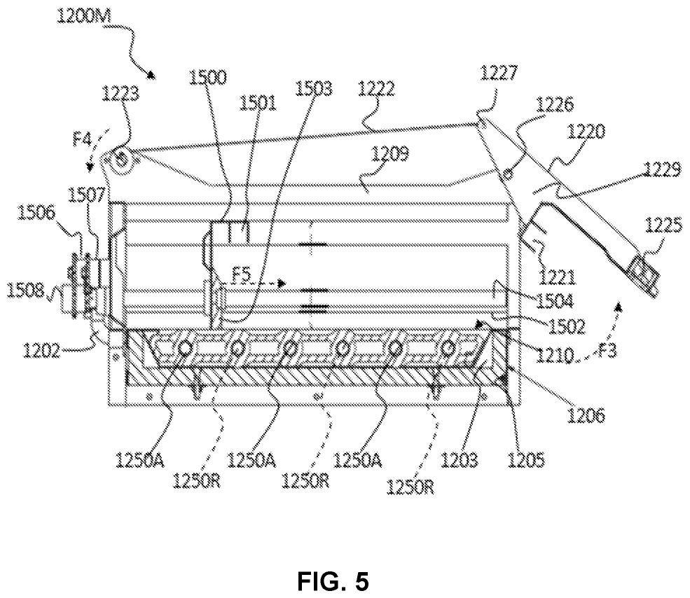

FIG. 5, a cross-sectional diagram of the module in FIG. 3,

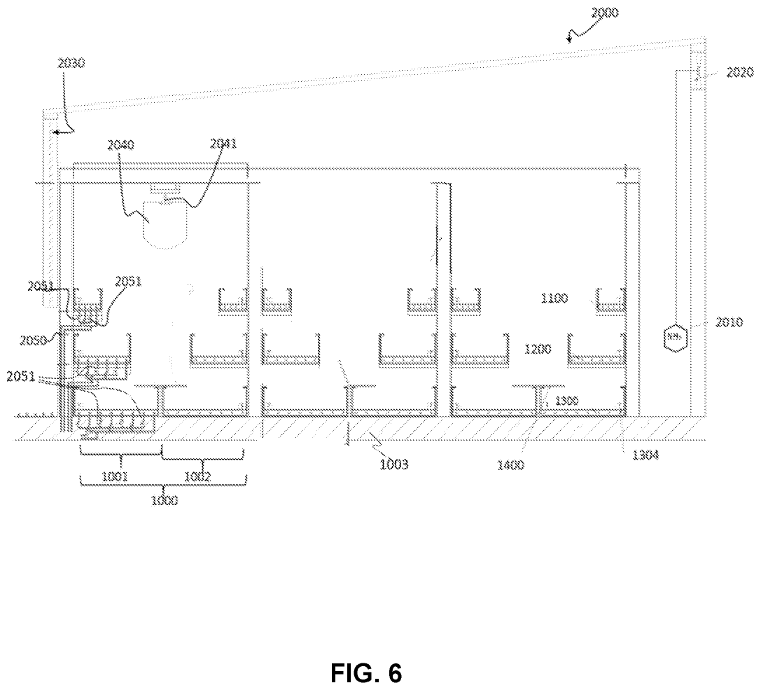

FIG. 6, a cross-sectional diagram of a building for farming insect larvae according to a preferred embodiment of the invention.

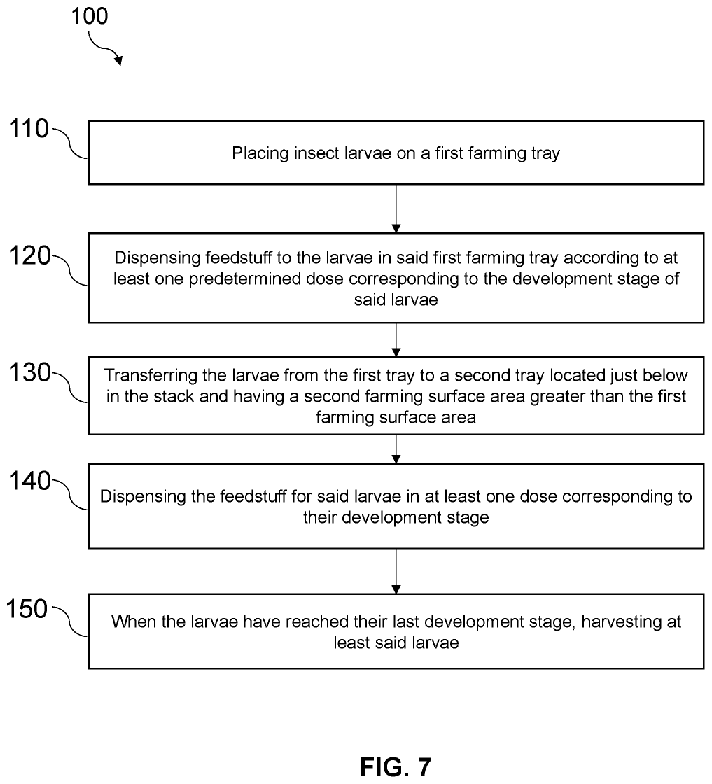

FIG. 7, a schematic representation of the insect larva farming method implemented in at least one farming unit according to the invention.

### DETAILED DESCRIPTION OF THE INVENTION

In the following description, by “side wall of a tray” is meant the vertical wall on one side of a tray, and more particularly the wall on the longitudinal side of a tray.

The term “mechanized” as used, refers to a device or system incorporating a mechanism for setting an element in motion.

The term “module” as used, refers to a juxtaposable element, combinable with other elements of a same nature, and contributing to a same function.

The term “transverse” as used, refers to an axis that runs through a space, perpendicular to its greatest dimension, that is to say perpendicular to its length.

The term “farming area/surface area” as used, refers to an area, in particular a surface area of a tray, intended for the growth of insect larvae. The farming surface area may correspond to part or all of the surface area of a tray suitable for larvae growth.

Advantageously, a farming unit in accordance with the invention comprises at least one row of at least two stacked trays.

Such a unit 1000 for farming insect larvae, as schematically shown in FIG. 1, comprises at least one row 1001 of at least two stacked trays. In the particular example illustrated in FIG. 1, a row 1001 comprises three overlapping trays referenced 1100, 1200, 1300. However, there is no limit to the number of stacked trays. This number is defined beforehand according to the growth cycle of the insect larvae to be farmed. Indeed, from the growth cycle of the larvae can be determined the number of development stages of the larvae. A tray will then be dedicated to a particular stage of development of the larvae. The stacked trays 1100, 1200, 1300 each have a total surface that can be divided into a farming area and a neutral area. Advantageously, the farming surface areas of the trays are of different dimensions and adapted to the development stage of the larvae they contain. Preferably, the trays have increasing farming surface areas and are arranged in a stepped manner, the tray with the smallest farming surface area being arranged at the top of the stack.

Indeed, the stack of the trays coupled with their thermoregulation allows to answer the problems generated by the conventional systems which involve obstacles to the thermal regulation by convection or the overconsumption of energy. Indeed, for a same larva production, a system according to the invention will have a more equivalent occupancy volume, aeraulics, and reduced energy consumption.

Table 1 below shows the results of the comparison of energy consumption between conventional facility and a facility according to the invention.

| Facility producing 15 tons per day of | Annual energy | Investment cost |
|---|---|---|
| larvae | cost (kWh) | (relative unit) |
| System according to the invention with | 3,850,000 | 10 |
| increasing thermoregulated surface | | |
| areas | | |
| Vertical system with stacked plastic | 25,100,000 | 100 |
| containers with thermal regulation by | | |
| ventilation | | |

Thus, there is indeed a significant energy gain with the present invention, coupled with a faster farming process. In particular, the farming units according to the invention allow a reduction by a factor of 6.5 compared to farming in containers with convection thermoregulation. In addition, the capital required for the construction of farming units according to the invention and the associated building is reduced by a factor of about 10.

In the preferred embodiment, illustrated in FIGS. 1 to 6, each tray has only one farming surface, that is to say the farming surface of a tray covers the entire surface of said tray. In this way, all surfaces are occupied by the larvae and there is no waste of unused surface.

Since the trays each have a farming surface adapted to a predetermined development stage of the larvae associated therewith, they remain advantageously fixed throughout the growth cycle of the larvae and it is the larvae that are moved from one farming tray to another during their development.

Thus, during the first stage of development, the smallest larvae are placed in the tray 1100 located at the top of the stack and, as soon as the larvae have reached the end of their first stage of development, they are transferred, by gravity, to the tray 1200 immediately below in the stack, said lower tray 1200 having a surface area greater than the surface area of the first tray 1100 and adapted to the second stage of development of the larvae, and so on up to the last stage of development and the last farming tray 1300.

Preferably, the ratio between the farming surface area values of two consecutive trays of the stack is between 2.2 and 1.2. More preferably, the ratio is between 2 and 1.5.

The length of the trays is the same for all trays in the stack. The trays are very long. Preferably, they are made in a modular way. In fact, the farming trays are comprised up of a plurality of farming modules connected to each other by their transverse wall, so as to form trays of a great length. This length, of several tens of meters, will depend on the quantity of larvae to be farmed. It could be between 10 and 300 m, for example. According to a particular embodiment, which is by no means limiting, the modules are for example 3 meters long each and 14 modules are connected to each other to form farming trays 42 meters long. A farming module, referenced 1200M, is shown schematically in FIGS. 3 to 5 in different views.

In order to have a surface area adapted to the development stage of the larvae, the width of the trays of the stack is adjusted, the width of each tray increasing as one goes down in the stack. The width of each tray of a stack of N trays can be expressed according to the following formula:

Iₙ=[(n)/N]*

[symbol]

wherein N is the total number of trays, I_{N} is the width of the last tray N located at the bottom of the stack, Iₙ is the width of a tray n, with n being between 1 and N, the tray of rank 1 being located at the top of the stack while the tray of rank N is located at the bottom of the stack. Thus, in the example shown in FIG. 1, the number of trays is 3 and the middle tray 1200 has a width I₂=(⅔)I₃, with I₃ being the width of the third and final tray 1300 of the stack. As regards the first tray 1100, it has a width I₁=(⅓)I₃.

Preferably, the trays are spaced from each other by a height of at least 10 cm, preferably at least 20 cm. This height corresponds to the space between the upper edge of the side wall of a tray and the lower wall (referenced 1211 in FIG. 4) of the tray immediately above. This space contributes, like other features of the present invention, to better growth of the larvae by acting on aeration and thermoregulation.

Preferably, an insect larva farming unit 1000 comprises two rows 1001, 1002 of at least two stacked trays each, arranged longitudinally opposite each other and separated by a floor 1400. Thus, having two rows of stacked trays by unit allows the production yields and the used farming surface areas to be optimized. The space between the two rows allows the passage of production personnel who can move on the floor located above the last tray and, favorably, of a larva feedstuff dispenser tank. Such a tank is described in more detail in the following description.

In a very advantageous embodiment, the last tray 1300 of each row, which has the largest surface area, is placed on the ground 1003 and under the floor 1400 separating the two rows 1001, 1002. In such a configuration, the floor 1400 separating the two rows is movably mounted so that it can open to allow access to the lower tray 1300.

To this end, a first embodiment of the movable floor, illustrated in FIG. 1, is to provide a floor mounted on a central beam 1430, the height of which is substantially flush with the upper end of the side walls of each lower tray 1300. The floor is then configured to be rotatable about an axis so as to be able to pivot and allow access to the lower tray 1300, particularly when dispensing the feedstuff, for example. This axis may, for example, take the form of a hinge and is preferably placed at the upper end of the central beam 1430 supporting the floor 1400, so as to articulate each longitudinal portion 1410, 1420 of the floor, located on either side of this central beam 1430. In the example illustrated in FIG. 1, each floor portion 1410, 1420 is pivotally mounted outwardly of the tray 1300, in the direction of the arrows referenced F1 and F2.

A second possible embodiment of the movable floor is to provide a translatably movable floor, as illustrated in FIG. 2. To this end, the transverse walls of the floor may be slidably mounted along guides referenced 1401, formed by rods for example and fixed substantially flush with the upper end of the side walls of each tray 1300. In this case, the floor 1400 separating two rows and located above each lower tray 1300, opens from the middle, each longitudinal part 1440, 1450 of the floor sliding along the guides 1401 in a translational movement towards the opposite longitudinal wall of the associated lower tray 1300, according to the direction of the arrows referenced F6 and F7 in FIG. 2, respectively.

Such a retractable floor has the advantage of allowing human intervention in case of need and allows an operator to reach without difficulty the trays 1100 located at the top of the stack for a possible intervention. The opening of the floor further allows access to the lower trays 1300 for a possible intervention and/or to allow gravity dispensing of the feedstuff by means of a dispenser tank.

Preferably, the trays are made of a metal, metal alloy, polymer, composite material, concrete, or a mixture thereof. Preferably, they are comprised of metal or a metal alloy. For example, they can be made mainly of metal or metal alloy. Nevertheless, they can advantageously include a combination of materials allowing to better thermoregulate the farming surface while consuming a minimum of energy.

As regards the last lower tray 1300, located at the bottom of the stack, when placed on the ground, under the movable floor 1400, it may comprise a concrete topping.

The farming units or, more generally, a system including a plurality of farming units according to the invention, may advantageously include one or more temperature measuring devices. These devices can for example be temperature probes, but also thermal imaging cameras. The temperature probes can be configured to measure the temperature of the ambient air, of the heat transfer fluids (for example at the tray inlet and/or outlet), of the growth surface areas, or of the trays.

For example, each tray is advantageously equipped with at least one temperature measuring means such as a temperature probe 1104, 1204, 1304, in order to monitor the temperature of the farming surfaces operated. Preferably, each tray comprises a plurality of temperature probes regularly arranged along its longitudinal axis, in order to monitor the temperature of each farming surface operated along its entire length.

Very advantageously, each farming tray is also equipped with a means for thermoregulating its used surface and thermoregulation of each tray is controlled independently.

To this end, each tray comprises a set of pipes 1150, 1250, 1350 configured to allow the circulation of a heat transfer fluid. Advantageously, the heat transfer fluid is regulated by a centralized unit not shown, consisting of either a heat pump or a thermorefrigerating pump, arranged in the farming building 2000 and allowing the heat transfer fluid to be circulated in all the pipes of each of the trays. The pipes are, for example, linear and may have circular cross-sections. Nevertheless, the pipes can take the form of a cavity positioned below the farming surface. Their number varies from one tray to another, depending on the width of said tray. When the constituent modules of each tray are connected to each other, their pipes are connected to each other by crimping or screwing, for example. At one end of the tray, opposite the heat transfer fluid inlet ports in the pipes, the consecutive pipes are connected two by two, by means of flexible hoses for example, so that a first pipe allows the heat transfer fluid to enter, and its neighbor allows the heat transfer fluid to return to the centralized unit, such as a thermorefrigerating pump, for example. Two consecutive pipes connected two by two thus form a pipe network. The hoses for connecting the ends of two pipes can be arranged inside the end module of the tray or outside the end module. In the example shown in FIGS. 1, 2 and 6, the upper tray 1100 comprises two pipe networks, while the middle tray 1200 comprises three pipe networks, and the lower tray 1300 comprises four pipe networks. As mentioned, these pipes could be replaced by a single cavity suitable for the circulation of a heat transfer fluid. In FIG. 5, the ports of the pipes allowing the entry of the heat transfer fluid are referenced 1250A, while the ports of the pipes allowing the return of the fluid to the centralized unit (thermorefrigerating pump for example) are referenced 1250R. The flow rate of the fluid circulating in each pipe is controlled in order to thermoregulate the surface of each one of the trays. To this end, each pipe of each tray may be equipped with at least one flow meter, not shown, intended to measure the value of the flow rate of the fluid flowing in the pipe and a solenoid valve, referenced 2051 in FIG. 6, placed upstream of each inlet port 1150A, 1250A, 1350A of each pipe network, is controlled by a control system to control the opening and/or closing thereof in such a way as to allow the fluid to flow at a predetermined flow rate to obtain a targeted temperature over the entire length of the farming surface area used or of the tray.

In a particular embodiment, the temperature control can be performed by positioning temperature probes upstream of each inlet and downstream of each outlet of the pipes, respectively, allowing the heat transfer fluid to enter/exit a given tray. Thus, it is possible to measure the inlet temperature of the heat transfer fluid, and then to measure the temperature thereof when it returns to the centralized unit and thus to control the temperature of each farming surface of each tray by controlling the flow of the heat transfer fluid as detailed above.

Alternatively, the temperature probes may be replaced by any other temperature measuring device known to the one skilled in the art and configured to provide temperature measurements of the farming surface of a given tray, of a farming unit according to the invention, or more generally of a farming building. Such other temperature measuring devices may be, by way of non-limiting examples, thermal imaging cameras.

Preferably, for weight reasons, the upper 1100 and middle 1200 trays are metallic and have a cavity, referenced 1203 in FIG. 5. The bottom of the cavity is covered with a thermal insulation material 1205. This material can for example be an insulating polymer such as polystyrene or polyurethane for example, mineral wool, cellulose wadding, expanded cork, bio-based wool, or any other equivalent material. This material allows the bottom and side walls of the cavity 1203 to be insulated in order to optimize heat exchange so that it is only towards the farming surface 1210. Advantageously, the cavity 1203 is filled with a heat-conducting liquid or material (for example concrete). The pipes 1250A and 1250R provided for the flow of the heat transfer fluid run through this cavity 1203 and are linear along the entire length of the tray. The cavity is covered by a metal plate constituting the farming surface area 1210 on which the larvae and their nutrient medium are placed. Thus, the flow of the heat transfer fluid through the pipes 1250A, 1250R allows for heat exchange with the farming surface area 1210 via the heat-conducting liquid or material filling the cavity 1203. By way of a non-limiting example, the heat-conducting medium filling the cavity can be glycolated water or oil for example or a material such as concrete or a metal such as aluminum, for example.

When the filler material is a liquid, its density varies with temperature. Therefore, in order to adjust the level of liquid in the cavity 1203, there is provided, on the rear wall of each constituent module of a tray, at least one adjustment pipe 1202, in fluid communication with the interior of the cavity 1203 and closed at the outer end thereof. This adjustment pipe 1202 allows the liquid to flow according to the principle of communicating vessels when the density of the liquid in the cavity increases, due to its temperature.

According to an alternative embodiment, the lower tray 1300, when placed directly on the ground 1003, and under the retractable floor 1400 for separating two rows, may advantageously comprise a concrete topping. In this case, the tray 1300 rests on a slab 1003, which may be a concrete slab, for example. The heat transfer fluid circulation pipes are secured on or in an insulating material. In one embodiment, the insulating material may, for example, take the form of an insulating studded plate of expanded polystyrene so that the pipes can be held in place by clipping them between two studs. Alternatively, the linear pipes can be fixed to a plate of insulating material by means of conventional fasteners, such as staples. A concrete topping is then poured to cover the pipes and the insulating plate and to create a smooth surface for larva farming. In this case, the heat transfer fluid flowing in the pipes allows a transfer of thermal energy with the concrete farming surface of the tray. In such an alternative, it is preferable to place the connection hoses of two consecutive pipes, to allow the return of the heat transfer fluid to the centralized unit (in the form of a thermorefrigerating pump or a reversible heat pump for example) outside the end of the last constituent module of the tray, so that they do not risk being damaged when the concrete topping is poured.

The farming unit advantageously includes a transfer device for mechanically and preferably automatically transferring the larvae from one tray to a lower tray. In particular, it is configured to transfer larvae and their substrate, an organic material consisting in particular of a feedstuff for the larvae as well as their waste, to a tray located just below in the stack. A mechanized transfer device according to the invention allows the farming surface area to be optimized according to the stage of development of the insect, the energy consumption for thermoregulation to be limited to the used surfaces only, this thermoregulation to be adjusted independently for each tray.

Very advantageously, the trays of the stack, or at least the trays 1100, 1200 located above the last tray 1300 of each stack, are equipped with a movable side wall, 1120, 1220, respectively, which is arranged to allow the transfer of the larvae, by gravity, into the tray, 1200, 1300 immediately below, respectively. Alternatively, the trays of the stack, or at least the trays 1100, 1200 located above the last tray 1300 of each stack, may be equipped with a larva retention means such as a stop or a rim.

A movable wall according to the invention may for example be the farming surface arranged to be inclined so as to cause the larvae to move by gravity towards a tray located below. A movable wall according to the invention can also be a vertical wall of a tray, with the movable wall being then arranged to move alone or as a bottomless crate to push the larvae and move them by gravity to a tray below.

A constituent farming module, referenced 1200M, of the middle tray 1200 and equipped with an elementary movable wall 1220, is illustrated in FIGS. 3 to 5 with its movable wall in the open position. The opening of the movable wall takes place in the direction of the arrow F3 in FIGS. 1, 2 and 5, so that the wall moves towards the outside of the tray.

The elementary movable wall 1220 of a farming module 1200M is, for example, movably mounted about a rotation axis 1226 materialized by two parts each connecting one of the two side uprights 1229 of the movable wall and one of the two transverse walls 1209 of the constituent farming module 1200M of the tray 1200. The movable wall can be rotated, in the direction of the arrow F3, using a shaft 1223 placed opposite the longitudinal wall opposite the movable wall and connected to the movable wall 1220 by means of two rigid rods 1222 or two straps, for example. Each rigid rod 1222 is attached at a point 1227 located at the upper end of one of the side uprights 1229 of the movable wall 1220. When the axis 1223 is rotated, according to the arrow F4 in FIG. 5, the rigid rods 1222 are in turn driven in movement around this axis 1223 and exert a tensile force at the points 1227 of attachment of the elementary movable wall 1220, rotating the movable wall around its axis 1226, according to the arrow F3, and causing the opening thereof.

In an alternative embodiment, when the rigid rods 1222 are replaced by straps, the rotation of the axis 1223 along the arrow F4 causes the straps to wrap around the axis 1223. The length of the straps then decreases, and the latter exert a tensile force at the points 1227 of attachment with the movable wall 1220, which rotates the movable wall 1220 about its axis 1226, according to the arrow F3, causing the opening thereof.

The axes 1223 of each constituent farming module 1200M of a tray 1200 are connected to each other, so that the actuation of one axis 1223 causes the actuation of the other axes 1223 of the other juxtaposed modules, and the elementary movable walls of all the constituent modules of the tray are integral with each other and open in a synchronized manner, so that they form a single movable wall of a great length.

The movable wall 1120, 1220 of each tray 1100, 1200 is provided, on its upper end located opposite the farming surface of the tray, with an inverted “U”-shaped rim, referenced 1221 in FIG. 5. This rim advantageously allows the crawling larvae to fall by gravity onto the farming surface, thus avoiding the escape thereof. For the same reason, the upper ends of the side walls of the trays 1100, 1200, 1300 are also provided with such an inverted “U”-shaped rim, referenced 1101, 1201, and 1301 in FIGS. 1 and 2, respectively.

In addition, closing means are provided on the movable wall to ensure that the farming surface is sealed when the movable wall is in the closed position. The closing means may for example be selected from magnets, electromagnets, cylinders, or latches. In particular, magnets 1225 are provided, evenly spaced along the lower portion of the movable wall 1220. When the movable wall closes, its lower portion is positioned against the outer edge of the cavity 1203. When the cavity is metallic, the magnets 1225 cooperate with the outer edge of the cavity to hold the movable side wall 1220 in a closed position. However, in a preferred embodiment, a magnetized plate 1206 is additionally placed against the inner wall of the cavity 1203 to further hold the movable wall 1220 in its closed position. A seal 1228, made of foam or silicone for example, may further be provided along the farming surface and on the outer edge of the cavity 1203.

The trays 1100, 1200, preferably equipped with a movable wall 1120, 1220, for example a movable side wall, are further advantageously equipped with a scraper 1500 configured to transfer, by gravity, the larvae, their nutrient medium, and residual frass to the tray immediately below, 1200, 1300, respectively, in the stack. This scraper 1500, which may also act as a movable wall, comprises a blade 1503 moving in contact, or substantially in contact, with the farming surface 1210 so as to move the larvae away from this farming surface. The scraper is moved in translation along the transverse axis of the tray as schematically shown in FIGS. 3 and 5 by the arrow F5. Preferably, guides 1502 are placed along each transverse wall of the tray so as to guide the scraper as it moves and so that it does not deviate from its path. The scraper 1500 may be used to transfer larvae over the larva retention means or the movable wall 1120, 1220, when in the open position. Advantageously, when the movable wall transitions from the closed position to the open position, the farming surface 1210 is transferred to the tray immediately below, by the scraper 1500, in a translation movement along the transverse axis of the tray.

Since the tray has a modular construction and is made by juxtaposing a plurality of modules, the scraper of a tray is in fact comprised of several elementary scrapers synchronized with each other.

Indeed, each constituent module of a tray is equipped with an elementary scraper along its entire length. When the modules are connected to each other to form a tray with a great length, the elementary scrapers of the tray are connected to each other, and a same control means controls them in a synchronized manner. Thus, if the elementary scraper of a constituent module of a tray fails, it does not prevent the other elementary scrapers of the other constituent modules of the tray from operating. In the example embodiment shown in FIG. 4 which illustrates an elementary farming module 1200M, the chain tensioning gears referenced 1507 of each module, allow chains to connect the elementary scrapers of each module to each other, so that all of the elementary scrapers are operated in a synchronized manner and form a scraper with a great length.

Different equivalent means can be used to actuate the translational movement of the scraper. This can be a rotation around endless threaded rods, referenced 1504 in FIGS. 3 and 5. In this case, the endless threaded rods 1504 are rotated via a drive chain referenced 1505 in FIG. 4, with the chain being tensioned by chain tensioning gears referenced 1506, and 1508. A gear 1508, associated with each threaded rod 1504, is connected to another gear 1507 via another chain, not shown and connected to the gear 1507 of an adjacent module. Thus, a single motor is used to drive a gear 1507 which drives the other gears 1507, 1508 and 1506 using a juxtaposition of chains between the various constituent modules of the tray 1200. The elementary scrapers of the various modules are thus operated in a synchronized manner, so that they form a single scraper of a great length.

According to other alternative embodiments, other equivalent means can be used to actuate the translational movement of the scraper, such as traction by means of cables or pushing the scraper by means of pistons.

Advantageously, the movable wall and the transfer device can be coupled and merged to form a transfer system similar to a bottomless drawer. In the closed position, the walls of the drawer allow to contain the larvae and their substrate on the tray. When the mechanized bottomless drawer moves across the tray to an open position, it allows the transfer of the larvae and substrate to the tray below.

Also advantageously, the trays are equipped with a movable wall along a length and a movable wall along a width. The movable wall can be opened across the width by a vertical hatch system. The opening allows the entry of an independent movable transfer system running on the tray. The movable transfer system moves longitudinally on the tray and has a transverse transfer means which can be a screw or a scraper to push the larvae and their substrate through the opening of the longitudinal movable wall of the tray, which is then in the open position, to the tray below.

As for the movable walls, the upper end of the scrapers is equipped with an inverted “U”-shaped rim 1501 allowing the crawling larvae to fall back onto the farming surface by gravity and thus avoiding that some larvae escape from their farming environment defined by a farming surface area 1110, 1210, 1310.

As regards the last tray 1300 in the stack, located at the bottom of the stack, it is not equipped with a movable wall. This tray is the farming tray for the larvae in their last stage of development. Preferably, this last tray 1300 is advantageously comprised of a single piece, that is to say without modular juxtaposition. At the end of this last stage of development, the larvae must be harvested, along with their nutritive medium, and the residual frass, to be conveyed to a processing unit.

Such harvesting of larvae can be done by means of a mechanized harvesting device, preferably an automated device. The mechanized harvesting device may be any means of transferring the larvae without damaging them from their place of growth to their place of processing. A mechanized harvesting device could be, for example, a screw conveyor, a belt conveyor, or a conveyor belt. In particular, the mechanized and automated harvesting device is preferably translatably movable along the longitudinal axis of said tray. Such a mechanized and automated harvesting device speeds up the harvesting process. In a favorable arrangement, the mechanized and automated harvesting device is a scraper-suction device, preferably translatably movable along the longitudinal axis of said tray, connected to a suction system via a network of hoses. In another arrangement, the mechanized and automated harvesting device is comprised of a movable longitudinal wall on the lower tray, a device for movably transferring the larvae and their substrate/windrow material through the movable wall to a conveyor device, which may be a belt, for transporting the harvested larvae and substrate to a centralized point. Such a mechanized and automated harvesting device speeds up the harvesting process.

This automated harvesting device may also correspond to a suction system coupled to harvesting means via a suction network equipped with pick-up points distributed throughout the farming unit, with the harvesting means being arranged to harvest the larvae from the trays located at the bottom of each stack.

However, said harvesting is preferable carried out by means of a scraper-suction device, not shown in the figures. This scraper-suction device has a length equal to the width of the tray 1300 and is moved in translation along the longitudinal axis of the tray. As it travels, the contents of the farming surface are sucked up by a hose or network of hoses, not shown, coupled to a suction system, not shown, and connected to the scraper-suction device. Each lower tray in each row can be equipped with such a scraper-suction device.

More preferably, a single scraper-suction device, in the form of a suction robot, is used to harvest the larvae from the lower trays. To this end, a transverse end of each lower tray 1300 is equipped with an opening, for example in the form of a guillotine flap, to allow the passage of the suction robot from one lower tray to another.

Also advantageously, the lower trays are equipped with a movable wall along a length and a movable wall along a width. The movable wall can be opened across the width by a vertical hatch system. This opening allows the entry of an independent movable transfer system running on the tray. The movable transfer system moves longitudinally on the tray and has a transverse transfer means which can be a screw or a scraper to push the larvae and their substrate through the opening of the longitudinal movable wall of the tray, which is then in the open position, to a conveyor system arranged along the tray below. Ideally, the conveyor system is a belt located between 2 lower lines and facing each other (2 lower trays of a same unit).

According to another embodiment, the trays of the farming unit each have a total surface area, a portion of which constitutes the larval farming surface area, and this farming surface area increases in accordance with the development stage of the larvae. A particular example of such an embodiment is to make the trays with identical dimensions. The trays are then divided along their length by removable dividers, such as hatches, for example. The length of each tray is then divided into as many parts as there are development stages of insect larvae to be farmed. Thus, only a first surface area, corresponding to the farming surface area, is used on a first part of the tray during the first stage of development of the larvae. Then, at the end of this first stage of development, a first removable divider is retracted, so as to increase the farming surface area for the second stage of development of the insect larvae. These steps are repeated until the farming surface area corresponding to the last stage of development of the insect larvae is used.

Advantageously, the thermoregulation means can then be modular and be configured to thermoregulate only a part of the surface area of the tray, corresponding to the farming surface area associated with a development stage of the larvae. Thus, at the first stage of development of the larvae, only the farming surface area corresponding to the first part of the tray is thermoregulated. The thermoregulated farming surface area thus increases in accordance with the development stage of the larvae, which allows an optimal temperature targeted for the development of the larvae to be maintained without risking to influence the development of the larvae in other trays. Indeed, as only the farming surface area is thermoregulated, an increase in temperature will only influence the development of the larvae present on said farming surface area. This is particularly advantageous since it is possible to grow larvae at different stages of development in different trays of a same farming unit at the same time, while optimizing the energy used to heat the farming surface area. The means for thermoregulating these farming surface areas is made in accordance with that described above with respect to the preferred embodiment of the farming unit according to the invention. Three-way valves, for example, arranged at the removable dividers of the tray, then allow to divert, or not, the heat-transfer fluid flowing in the pipes of the thermoregulation means, in order to redirect it, or not, towards the thermorefrigerating pump, for example.

Of course, the farming unit according to this embodiment may include a plurality of trays which may be stacked.

A larva feedstuff dispenser tank, controlled by a feeding control device, allows to convey at regular intervals, doses of feedstuff to the used farming surface areas, as a function of time.

The tray also comprises a scraper-suction device, translatably movable along its longitudinal axis when all the removable dividers are in the retracted position. This scraper-suction device is connected to a suction system by a hose or a network of hoses and allows the larvae to be harvested at the end of their last stage of development, in order to transfer them to a processing unit.

Not thermoregulating the ambient air but only the farming surface areas in contact with the larvae allows the energy requirements of thermoregulation to be optimized. Finally, the farming trays are not intended to be moved, they remain in place and their farming surface is constantly thermoregulated independently. The larvae are fed at regular intervals by means of a feeder such as a feedstuff dispenser tank which is conveyed to the farming surface areas by means of a control program.

FIG. 6 shows a cross-sectional diagram of an insect larva farming building 2000 comprising three insect larva farming units 1000. This diagram illustrates only one possible embodiment of the invention. Indeed, the invention is not limited to this embodiment, as the number of larva farming units and the number of farming trays per unit, in particular, may be modified according to the nature of the insect larvae to be farmed therein. Similarly, the building may comprise farming units according to the first preferred embodiment and/or farming units according to the second embodiment.

The particular example shown in FIG. 6 was developed for farming Hermetica Illucen larvae, known as the black soldier fly. The number of farming units, according to the second embodiment, and of trays per unit has been previously determined according to its development cycle. The optimal growth period for this insect is 9 days. This growth cycle allowed the identification of three distinct development stages of three days each. One farming tray 1100, 1200, 1300 was thus reserved per stage of development. With this growth cycle, a larva farming unit 1000 allows for one larva harvest every three days. By placing three farming units 1000 in the farming building 2000, it is possible to harvest once a day. This example is therefore specific to one type of insect and the one skilled in the art will know perfectly well how to adapt the invention and, in particular, how to modify the number of units in the building and the number of trays per unit according to the development cycle of the insect larvae to be farmed.

The building 2000 dedicated to farming insect larvae comprises at least one farming unit 1000. Preferably, it comprises a plurality thereof arranged side by side. In the particular example of farming black soldier fly larvae, the building preferably comprises three units, each unit comprising two rows 1001, 1002 of three trays 1100, 1200, 1300 stacked on top of each other, so as to allow a daily harvest of larvae.

In this particular example of farming black soldier fly larvae, if the farming units of the building are constructed according to the second embodiment, then each unit may comprise at least two trays, with each tray being divided into three parts along its length.

Even if the farming surface of each farming tray is thermoregulated independently, it is also necessary to deconcentrate the ambient air of the building from the surplus of gases including ammonia released by the larvae and their substrate during farming and to regulate the hygrometry of this air. To this end, the building is equipped with conventional extractor turbines 2020 regularly arranged on one of the longitudinal walls of the building. These extractors are preferably arranged at the top of the longitudinal wall.

The building also comprises at least one sensor for measuring the concentration of ammonia in the gas phase and the hygrometry, referenced 2010 in FIG. 6. The result of the one or more measurements taken by the probe(s) is communicated to an aeration control device which activates the extractors and rotates them more or less rapidly depending on the amount of ammonia and humidity to be evacuated.

The building also has a closable air inlet 2030 on the longitudinal wall opposite the one equipped with the extractor turbines. This reclosable air inlet can for example be in the form of a flap curtain. This curtain is also controlled by the aeration control device, in order to open the curtain flaps more or less depending on the amount of fresh air to be brought into the building. Thus, the faster the turbine of the extractors 2020 rotates, the greater the opening of the flaps to renew the air in the building.

The new air entering the building is not thermoregulated, since the thermoregulation is carried out only on the used surface of each tray, directly in contact with the larvae, and adapted to the development stage of the larvae. Thus, the energy consumption for thermoregulation is considerably reduced compared to existing installations, which consist of thermoregulating the entire volume of air entering the building, even though it is extracted from the building almost instantaneously. Indeed, this thermoregulation system favors a better thermal exchange surface with the larvae.

A network of pipes 2050 allows a heat transfer fluid to be circulated between a centralized unit, in the form of a reversible heat pump or a thermorefrigerating pump, and the pipes 1150, 1250, 1350 of each tray of each unit. The building also comprises a control unit for controlling each thermoregulation means of each farming tray of each unit independently, according to the stage of development of the larvae, temperature values measured on the used surface of each tray, and measured values of the flow rate of the heat-transfer fluid flowing in the pipes 1150, 1250, and 1350 of each tray. To this end, the control means controls the opening and/or closing of each solenoid valve 2051, placed opposite each pipe inlet port 1150A, 1250A, 1350A, in order to adapt the heat transfer fluid flow rate and to reach the target temperature of each tray. In this way, the amount of energy spent on thermoregulation is controlled.

The building also comprises a mechanized and preferably automated larva feeder. The mechanized larva feeder may, for example, take the form of a tank 2040 for dispensing larva feedstuff, with the contents of this tank being conveyed at regular intervals to the trays for feeding the larvae by means of a control program. Advantageously, the larva feeder, such as the tank, is suspended on a rail 2041, along which it moves, between the trays, and thus conveys the feedstuff. To this end, an automated means is used to control the movement of the larva feeder along the rail between the trays and thus to convey the feedstuff automatically and regularly, as a function of time, into the trays to enable feeding of the larvae. Upon passage of the larva feeder, the floor 1400 is automatically moved to be opened, upon control of the larva feeder controller (for example dispenser tank), to allow the feedstuff to be dispensed to the lower tray(s) placed below the floor. The rail is configured to allow movement of the larva feeder (for example tank) through all units of the building. Thus, the track includes turns outside the units so as to allow conveying of the larva feeder (for example tank) from one unit to an adjacent unit and so on.

When the farming units are in accordance with the second preferred embodiment, a further automated device, also referred to as a larva transfer control device, controls the opening and closing of the movable walls 1120, 1220 of the upper trays of each unit, independently. This automated device also makes it possible to control, in a synchronized manner with the opening of a movable wall of a tray, the transfer device such as the scraper of the corresponding tray. The control of the opening of a movable wall and of the transfer device such as the associated scraper can be carried out as a function of measured temperature values, for example on the surface of the tray, of the amount of feedstuff dispensed, and/or as a function of time. For example, a larva transfer device may be configured to take into account predetermined temperatures or predetermined times.

The building further comprises a suction system, not shown, coupled to a hose or network of hoses. The hose(s) is (are) connected to at least one scraper-suction device. A larva harvesting control device then makes it possible to activate the suction and the translational movement of the scraper-suction device along the longitudinal axis of a lower tray 1300, so as to allow the larvae to be harvested at the end of their last development stage.

Preferably, the devices for controlling the aeration of the building, for thermoregulating each tray, for transferring the larvae from one tray to another by actuating a scraper and the movable wall of the associated tray, for conveying and dispensing the feedstuff, and the scraper-suction device for harvesting the larvae are all combined in one and the same automatic supervision device. The temperature, ammonia concentration and hygrometry probes, and the flow meters are directly connected to this automatic supervision device in order to send thereto the measured values to enable it to adapt the control of the various elements. A clock is also connected to the automatic supervision device, or integrated into the device, for knowing the development stage of the larvae. The automatic supervision device thus makes it possible to activate the heat transfer fluid pump and the solenoid valves of each thermoregulation means, the extractor turbines, the reclosable air inlet, the conveying of the feedstuff dispenser tank, the scrapers and the movable walls, the scraper(s)-suction device(s), and the suction system.

The insect larva farming method 100, described in connection with FIG. 7, implemented by means of the farming units 1000 according to the first preferred embodiment advantageously comprises the following steps:
Placing 110 insect larvae on a first farming tray 1100,
Dispensing 120 larva feedstuff into said first farming tray,
Transferring 130, after a predetermined period of time corresponding to a first development stage, the larvae from the first farming tray 1100 to a second tray 1200 located just below the first farming tray 1100 and having a second farming surface area greater than the first farming surface area,
Dispensing 140 feedstuff to said larvae in at least a predetermined dose corresponding to their development stage, and waiting for the end of a second predetermined time corresponding to a second development stage, and repeating the steps of transferring the larvae and dispensing feedstuff to the larvae until the larvae are transferred and the feedstuff is dispensed to the larvae in the last tray at the bottom of the stack, and
when the larvae have reached their last development stage, harvesting 150 at least said larvae.

Advantageously, the insect larva farming method may also include a step of thermoregulating the trays, which may include a prior measurement of the temperature, for example at the trays, the growth surfaces, the heat transfer fluid, or the ambient air. Temperature probes can continuously transmit the temperature of the windrow to an automatic supervision device that can control the thermoregulation system. The average temperature of the windrow is preferably maintained between 25 and 35° C. In order to control this temperature, a thermostatic source is induced under the surface of the tray on which the windrow will rest. This surface must therefore be able to transmit the heat flow between the thermostatic source and the windrow. The estimated heat flux generated by the larvae is 150 to 200 W/m². Thus, thermoregulation via the tray is particularly advantageous.

Advantageously, and for better larva growth performance, the windrow can be stirred daily. The purpose is to oxygenate the windrow and release water vapor trapped in the lower layer of the windrow as well as to release any ammonia (NH₃) that may have formed to prevent its accumulation. Thus, the process may include the use of a stirring device configured to perform a delumping of the clusters formed by heterogeneous drying of the windrow (for example from a size of the order of 5 cm and more to a size below 1 cm), and/or a turning over of the upper layer to the lower layer, and vice versa.

In particular, a predetermined amount of insect larvae are arranged on a first farming tray 1100, located at the top of the tray stack and having a first farming surface.

Dispensing feedstuff to the larvae in said first farming tray is preferably done according to at least one predetermined dose corresponding to the development stage of said larvae. This dispensing can be done by means of a controllable and automated feedstuff feeder such as a feeder tank conveyed at regular intervals by means of a control program of a feeding control device. Preferably, the method involves at least daily feeding. For example, young larvae are grown in the first stage, shown for example by a farming tray 1100. After a period of 3 days, the larvae are transferred by a gravity mechanism to the lower tray 1200.

The feedstuff for said larvae is dispensed on the lower tray in at least a dose corresponding to their development stage, by means of said feeder tank conveyed at regular intervals via the control program of said feeding control device and wait for the end of a second predetermined time corresponding to a second development stage, and repeat the steps of transferring and feeding the larvae until the larvae have been transferred and fed in the last tray 1300 located at the bottom of the stack. In particular, after 3 days in stage 2 shown by the farming tray 1200, the larvae will be sent to stage 3 shown by the farming tray 1300 using the same gravity mechanism.

After 3 days of growth in stage 3, the larvae as well as their waste and food residues will be harvested by a harvesting system to be designed as well.

Advantageously, there are no daily production breaks—each stage is operated daily. The farming surface areas are thermoregulated. The floors on which the larva grow will be thermally regulated by a heat transfer fluid that will raise or lower the temperature of the trays.

Thanks to the invention, the ambient air, which is constantly renewed to remove the ammonia released by the larvae and its nutrient medium, is not directly thermoregulated. Only the farming surface area of the different trays directly in contact with the larvae is individually and independently thermoregulated. As a result, farming insect larvae in this way is much less energy intensive than in existing facilities. The farming areas are also sized according to the stage of development of the larvae, so that no surface area is wasted and the thermoregulated surface areas are all used.

1. A unit for insect larva farming including at least one row of at least two stacked trays, said unit being characterized in that said trays have farming surface areas of different dimensions, said farming surfaces being of increasing dimensions and are arranged in a stepped manner, the tray with the smallest farming surface area being arranged at the top of the stack, and in that each tray, located above the last lower tray of the stack, comprises a movable wall arranged to allow the transfer of larvae, by gravity, into the tray immediately below, and in that each tray comprises a means for thermoregulating its farming surface area.

2. The unit according to claim 1, characterized in that each tray is a tray thermoregulated by a heat transfer fluid.

3. The unit according to claim 1, characterized in that the movable wall corresponds to the farming surface arranged in a manner inclinable so as to cause the larvae to move by gravity towards a tray located below or the movable wall is a vertical wall of a tray, the movable wall being then arranged to move alone or as a bottomless crate to push the larvae and move them by gravity to a tray below.

4. The unit according to claim 1, characterized in that the means for thermoregulating operates by radiation and said farming surface is arranged so as to be directly in contact with insect larvae.

5. The unit according to claim 1, characterized in that the ratio between the farming surface area values of two consecutive trays of the stack is between 1.2 and 2.2.

6. The unit according to claim 1, characterized in that each tray is divided by removable dividers into as many parts as there are development stages of the insect larvae to be farmed, and in that the removable dividers are retracted as the stages of development of the larvae progress so that the farming surface area of the tray increases with the development stage of the larvae.

7. The unit according to claim 1, characterized in that it comprises two rows arranged longitudinally opposite each other, separated by a floor, each row comprising at least two stacked trays.

8. The unit according to claim 7, characterized in that the floor is movably mounted.

9. The unit according to claim 1, characterized in that each row comprises a stack of at least two trays, the last tray with the largest surface area being arranged on the ground (1003).

10. The unit according to claim 1, characterized in that the movable wall comprises closing means, for example magnetized means, ensuring the farming surface is sealed when said movable wall is in a closed position.

11. The unit according to claim 1, characterized in that each tray, located above the last lower tray of the stack, further comprises a translatably movable scraper, capable of transferring larvae to a tray located just below in the stack.

12. The unit according to claim 1, characterized in that the last tray of the stack comprises a mechanized harvesting device, preferably translatably movable along the longitudinal axis of said last tray.

13. The unit according to claim 1, characterized in that the last tray of the stack comprises a scraper-suction device, preferably translatably movable along the longitudinal axis of said tray, connected to a suction system via a network of hoses.

14. The unit according to claim 1, characterized in that said unit comprises at least one temperature measuring device, preferably configured to measure the temperature of the farming surface of each tray.

15. The unit according to claim 14, characterized in that the temperature measuring device is a temperature probe and in that each tray comprises at least one temperature probe.

16. The unit according to claim 1, characterized in that the thermoregulation means comprises pipes arranged linearly and configured to allow the flow of a heat transfer fluid, said pipes being arranged in a cavity, provided in the tray and filled with a heat-conducting liquid or material, said heat transfer fluid allowing a transfer of energy with the farming surface via said heat-conducting liquid or material.

17. The unit according to claim 1, characterized in that the thermoregulation means comprises linearly arranged pipes configured to allow the flow of a heat transfer fluid, said pipes being covered by a concrete topping forming the farming surface with which the heat transfer fluid performs an energy transfer.

18. A building for insect larva farming, characterized in that it comprises at least one insect larva farming unit according to claim 1.

19. The insect larva farming building according to claim 18, characterized in that it comprises a thermoregulation control device configured to control a thermoregulation means of each tray of each unit for insect larva farming, independently, as a function of temperature values measured on the farming surface of each tray and of flow rate values of the heat transfer fluid flowing in the pipes of said thermoregulation means.

20. The building according to claim 18, characterized in that it further comprises a tank for dispensing larva feedstuff and a feeding control device, configured to control the conveyance of the contents of said tank to the trays of each unit for insect larva farming, as a function of time.

21. The insect farming building according to claim 18, characterized in that it further comprises a larva transfer control device, configured to control the opening and closing of a movable wall of one or more trays of each unit for insect larva farming and to control, in a manner synchronized with the opening of said movable wall, a scraper of the corresponding tray and in that the control of the opening of the movable wall and of the scraper is carried out as a function of temperature values measured on the farming surface of each tray, of the amount of feedstuff dispensed, and as a function of time.

22. The insect farming building according to claim 18, characterized in that it further comprises a device for controlling the harvesting of mature larvae which is capable, on the one hand, of actuating a suction system coupled to a hose or to a network of hoses connected to a scraper-suction device of a lower tray and, on the other hand, of actuating said scraper-suction device in translation along the longitudinal axis of said associated lower tray.

23. The insect farming building according to claim 18, characterized in that it further comprises an aeration control device capable of activating the operation of extractors and the opening or closing of a reclosable air inlet, as a function of values recorded by at least one device for measuring the concentration of ammonia in the gas phase and the hygrometry level.

24. The insect farming building according to claim 18, characterized in that the devices for controlling thermoregulation, for controlling the transfer of larvae, for controlling the harvesting of larvae, for controlling aeration and/or for controlling feeding are combined in a single automatic supervision device.

25. A method of farming insect larvae implemented in at least one farming unit for insect larva farming including at least one row of at least two stacked trays, said trays having farming surface areas of different dimensions, said farming surfaces being of increasing dimensions and are arranged in a stepped manner, the tray with the smallest farming surface area being arranged at the top of the stack and in that each tray comprises a means for thermoregulating its farming surface area, said method being characterized in that it comprises the following steps:
placing insect larvae on a first farming tray, located at the top of the stack of trays and having a first farming surface, and dispensing larva feedstuff in said first farming tray in at least one dose corresponding to the development stage of said larvae, preferably by means of a feeder tank and at regular intervals by means of a feeding control device,
after a predetermined period of time corresponding to a first farming stage, transferring the larvae from the first tray to a second tray located immediately below in the stack and having a second farming surface area greater than the first farming surface area, said transfer implementing a movable wall arranged to allow the transfer of larvae, by gravity, into the tray immediately below,
dispensing the feedstuff for said larvae in at least one dose corresponding to their development stage, preferably by means of said feeder tank and at regular intervals, and waiting for the end of a second predetermined period of time corresponding to a second development stage, and repeating the steps of transferring and feeding the larvae until the larvae have been transferred and fed in the last tray located at the bottom of the stack, and
when the larvae have reached their last development stage, harvesting at least said larvae.
